(19) United States
(12) Patent Application Publication
(10) Pub. No.: US 2022/0175619 A1
(43) Pub. Date: Jun. 9, 2022

(54) MEDICATION DELIVERY APPARATUS AND SYSTEM

(71) Applicant: DOSELL AB, Stockholm (SE)

(72) Inventor: Göran SJÖNELL, Lidingö (SE)

(73) Assignee: DOSELL AB, Stockholm (SE)

(21) Appl. No.: 17/603,492

(22) PCT Filed: Apr. 15, 2019
(86) PCT No.: PCT/EP2019/059697

§ 371 Date: Oct. 13, 2021

(51) Int. Cl. A61J 7/04; A61J 1/03; A61J 7/00; G16H 20/13

(52) U.S. Cl. CPC A61J 7/0454; A61J 1/03; A61J 7/0436; A61J 7/0418; A61J 7/0076; A61J 2205/60; A61J 2200/74; A61J 2200/72; A61J 2200/70; A61J 2205/10; A61J 2200/30; G16H 20/13

(57) ABSTRACT

A medication delivery apparatus, including: a housing configured to accommodate a strip of medication packages in its interior and having an outlet for outputting medication packages from the interior of the housing; a lid of the housing configured to enable access to the interior of the housing; and an electronically controllable lock mechanism positioned in the interior of the housing and configured to lock the lid in a closed position. The apparatus is configured such that the lock mechanism is configured to be controlled from the exterior of the housing by a control device.

### TECHNICAL FIELD

The present disclosure relates generally to apparatus and systems for medication delivery to a patient. More particularly, the present disclosure relates to a system with medication delivery apparatus that are configured for delivering medication for a dedicated patient from a medication dose roll with a strip of medication packages.

### BACKGROUND

In order to help patients to take medication in prescribed dosages at scheduled time intervals there are pharmaceutical services providing prepackaged and labelled medication packages. These are often delivered on a medication dose roll with a strip of medication packages, each package having a label that may contain information on the specific patient, the content of medication in the package and the time when the dosage is prescribed to be consumed. Each package then contains the medication, for example a combination of one or more pharmaceuticals, which should be taken at a specific occasion in a prescribed dosage regime.

On the market, there are mechanized and computer controlled medication delivery apparatus, also called medication dispensers, dosage devices or the like. Such apparatus are devised to receive and house a medication dose roll and to output a medication package at certain points in time according to a selected schedule. The pharmaceuticals are stored inside the medication delivery apparatus and are not accessible by the patient, except for a medication package that is output for consumption the patient.

### RELATED ART

The patent publication EP 2 680 177 A1 to Evondos Oy shows an example of a medication dispenser and methods for controlling the output of medication packages based on information on the label of medication packages and patient information stored in the medication dispenser.

### OBJECT OF THE DISCLOSED EMBODIMENTS

The general object of embodiments of the present disclosure herein is to provide a medication delivery apparatus that enables improved patient safety.

### SUMMARY

Embodiments disclosed herein pertains to a medication delivery apparatus that comprises a housing configured to accommodate a strip of medication packages in its interior and having an outlet for outputting medication packages from the interior of the housing. A lid of the housing is configured to enable access to the interior of the housing. An electronically controllable lock mechanism is positioned in the interior of the housing and configured to lock the lid in a closed position. The lock mechanism is configured to be controlled from the exterior of the housing by means of a control device.

In embodiments the lock mechanism is configured to be unlockable only from the interior of the medication delivery apparatus, and may be configured such that it is operated and/or made accessible only from the interior of the housing, for example by means of a pre-programmed or remote controlled control unit comprised in the medication delivery apparatus.

In embodiments, the medication delivery apparatus is provided with:
a compartment configured to accommodate a strip of medication packages, each package being provided with indicia indicating time for consuming the medication of the package;
a medication package feeder mechanism configured to transport medication packages from the compartment to an outlet of the medication delivery apparatus;
a reader configured to read said indicia of a medication package being transported by the medication package feeder mechanism;
a control unit configured to control the medication delivery apparatus to output a medication package via the outlet at a time corresponding to the time for consuming as indicated by said indicia, and comprising a sensor configured to detect removal of an output medication packet from the outlet, wherein the medication delivery apparatus is configured to activate an alarm signal when a preset time period from the indicated time for consuming has passed without the sensor having detected that an output medication package has been removed from the outlet.

The reader is, in embodiments, a camera and the indicia is configured to indicate time for consuming the medication of the package is information printed on a label of the medication package. In embodiments, the control unit has access to information of a time schedule for outputting medication packages and wherein the medication delivery apparatus is configured to activate an alarm signal if the time for consuming the medication indicated on a medication package deviates from the corresponding time schedule information.

In embodiments the medication delivery apparatus further comprises or is communicatively coupled to one or more sensors arranged to provide patient specific data. The sensor may be one or more of a weight sensor, a motion sensor, a temperature sensor, proximity sensor, a temperature sensor (46), a continence sensor, a blood pressure sensor and/or a blood glucose sensor.

Embodiments of the medication delivery apparatus may further be configured to:
control the medication delivery apparatus to output one or more medication packages via the outlet at a predetermined time, possibly at least partly based on indicia of the one or more medication packages; and/or
compare patient specific data from one or more sensors with one or more pre-stored or calculated patient specific data values; and/or
generate an alert signal if said provided patient specific data deviates from said one or more predetermined patient specific data values; and/or
deliver medication packages with a predetermined time interval, and wherein said time interval is adjusted based at least partly based on sensor data and/or if an alert signal is generated; and/or
deliver a predetermined number of medication packages, wherein the number of medication packages is adjusted at least partly based on sensor data and/or if an alert signal is generated.

Embodiments comprise a medication delivery system, having a medication delivery apparatus configured to house a strip of medication packages; and a back end monitoring and/or control system; wherein the medication package strip storage of the medication delivery apparatus may only be accessed by control from the exterior of the medication delivery apparatus by means of a control device. Such embodiments, may be in the form of a medication delivery system, further comprising a selection of the above mentioned features.

### BRIEF DESCRIPTION OF DRAWINGS

Embodiments of the present disclosure will be explained below with reference to the enclosed drawings, wherein:

FIG. 1 shows a medication dose roll with a strip of medication packages filled with pharmaceuticals;

FIG. 2 shows an embodiment of a medication delivery apparatus for pharmaceuticals in a first state of operation;

FIG. 3 shows the embodiment of FIG. 2 in a second state of operation.

FIG. 4 shows embodiments of a medication delivery apparatus communicatively coupled to other devices by means of a data communications network.

### DETAILED DESCRIPTION OF EMBODIMENTS

Mechanized medication delivery apparatus are generally configured to receive and store a strip of medication packages in a roll. In operation, the machine transports the strip of medication packages in path to an outlet where a medication package is output and served to a patient or other user.

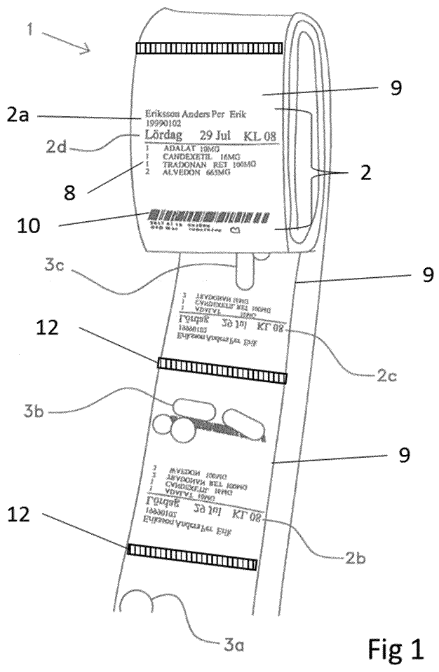

FIG. 1 shows a strip 1 of medication packages with pharmaceuticals or pharmaceutical preparations 3a-c, such as pills, packed in medication packages 9 with different packages 9a-c shown in different positions within the apparatus. In FIG. 1 the medication package strip 1 is configured in a medication dose roll. In other configurations the medication package strip 1 may for example be folded in an accordion like manner. The medication packages may be in the form of bags, pouches or sachets for example made in plastic material such as cellophane, paper or other suitable material. Each medication package 9 of the roll is provided with indicia 2 for example in the form of a label with information printed thereon. The indicia or the labels may be adhered to the medication packages or may be an integral part of the packages. Typically, the information comprises a selection of patient specific information such as the name of the patient 2a, the personal identity number or an equivalence such as a national identification number or social security number as well as a specification of the pharmaceutical contents 8 of the package and other production or packing data.

This information may further indicate at what point in time, i.e. date and time of day 2b-d, the medication package should be consumed or output from the medication delivery apparatus according to a dosage regime, to a schedule for taking the pharmaceuticals or to a schedule for outputting a medication package. In practical embodiments, the point in time indicated for consumption may be interpreted and/or approximated as the time for outputting the medication package, or vice versa.

A selection of the mentioned information and possible additional information may also be provided as machine readable indicia 10 on the medication packages for example in the form of a printed bar code, as exemplified in FIG. 1, or a QR code or as an RFID tag or a NFC tag.

Generally, it is preferred that the medication packages are provided with indicia indicating a selection of the information in plain text that is directly readable by human users as well as indicia in a machine readable format, although it is conceivable to have either human readable plain text or machine readable indicia. Plain text may be visual characters and/or protruding or recessed relief characters form Braille text. The medication packages are each provided with its individually applicable indicia, one set of indicia per package indicating the relevant information for its content.

Each medication package is separated from the next package of the strip with a package sealing 12, for example a heat sealing in the case of plastic medication packages or a glued sealing in case of paper medication packages.

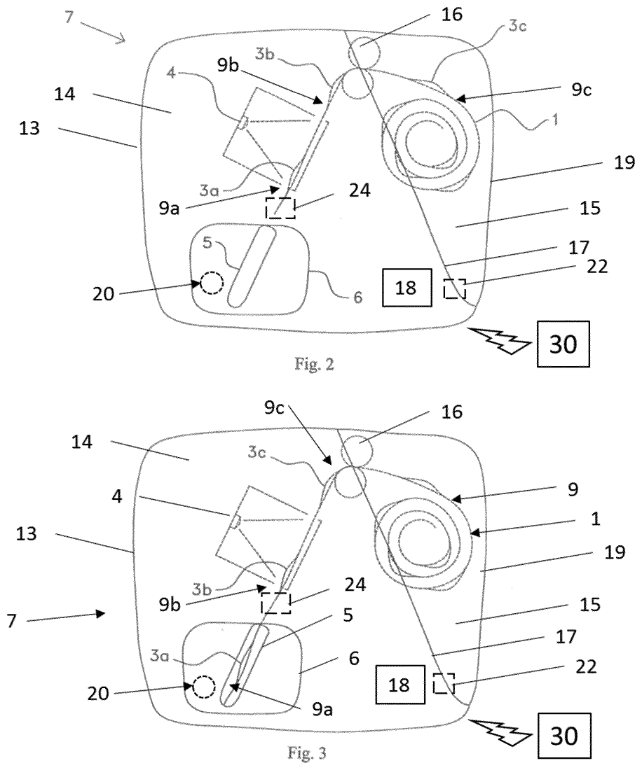

FIG. 2 shows an embodiment of a medication delivery apparatus 7 for pharmaceuticals in a first state of operation loaded with a strip 1 of medication packages 9 (9a-c) on a medication dose roll, each medication package packed with a set of pharmaceuticals 3a-c. In the shown first state of operation of the medication delivery apparatus the medication packages are all concealed in the interior and none is accessible to the patient.

The medication delivery apparatus 7 has a housing 13 which is in its interior comprises two separate compartments. A first compartment 14 is configured to accommodate machinery in the form of mechanical, electrical and electronic circuitry components, and a second compartment 15 is configured to accommodate a strip of medication packages on a medication dose roll 1.

The machinery compartment 14 accommodates machinery comprising a medication package feeder mechanism (16) configured to transport medication packages from the compartment 15 where the medication roll is positioned along a medication package strip path to an outlet 5 of the medication delivery apparatus. There is a reader 4 provided to read indicia on the medication packages 9a-c as they pass along the medication strip path. Close to the end of the path there is a cutting mechanism 24 configured to cut a medication package open just before it is output through the outlet 5.

The outlet 5 of the medication delivery apparatus is configured to receive a medication package 9 from the interior and output the medication package through a slot in the housing 13. The slot may be provided with a stop shoulder to prevent a medication package to fall out of the slot and with a recess to make the medication package stay in place until a patient or other user has picked the package from the outlet. Preferably there is a wider recess 6 in the housing 13 surrounding the slot of the outlet 5, the recess 6 giving space and allowing the user to seize a medication package with the fingers.

In the state shown in FIG. 2, a first medication package 9a of a medication package strip 1 is in a position exposed to the reader 4 such that the reader can read indicia of the package 9a, a second medication package 9b is in the path between the medication package feeder mechanism 16 at the inlet to the machinery compartment 14 and a third medication package 9c together with other medication packages on the roll of the strip 1 of medication packages in the compartment 15.

FIG. 3 shows the same medication delivery apparatus 7 in a second state, where the first medication package 9a has been output and is in a serving position in the slot of the outlet 5, the second medication package 9b is in a position for reading the indicia and the third medication package 9c in the path between the reader 4 and the medication package feeder mechanism.

In embodiments, the medication delivery apparatus comprise electronic circuitry components including a control unit 18 with a digital data processor having a memory and one or more of a communication equipment for data communication via wired or wireless internet such as with a wifi communication unit, telecommunication such as with a GSM unit with a SIM, short range radio communication (e.g. Bluetooth) or infrared light with external units such as an external database, sensors or other periphery equipment. The reader 4 may for example be a camera coupled to the data processor of the control unit 18 and the data processor may be equipped with an image processing system configured interpret indicia in plain text OCR (Optical Character Recognition) technology, bar code or QR code interpreting technology. In other embodiments, the reader may be an RFID reader configured to read and interpret indicia on an RFID tag or an NFC configured to read and interpret an NFC (Near Field Communication) chip.

The control unit 18 is in embodiments configured to control the medication package feeder mechanism 16 and the cutting mechanism 24 dependent on indicia 2 read from the respective medication packages 9. In one embodiment, the control unit 18 is configured by means of computer program code to output a medication package only at the point in time indicated on the medication package. The control unit 18 may be provided with an internal clock or be configured to retrieve and/or receive information on the current time from external sources, for example from a telecommunication system.

The computer system may also have access to separate information other than that indicated by the indicia of the medication packages about the time for the patient to consume the pharmaceuticals in a medication package and/or the time for outputting the medication package and giving the patient access to the pharmaceuticals. This separate information may be input and stored in the medication delivery apparatus by an operator or the control unit 18 may be configured to retrieve the information from an external database. The control unit 18 may be configured to compare time for consumption or outputting the medication package of the indicia of the package with said separate information, and if the two pieces of information do not match the medication package will not be output. In such embodiments, a medication package is output at the relevant point in time only if the two pieces of information are matching. Other embodiments are configured to operate based only on the information of the indicia of, i.e. on or tagged to, the medication packages.

An embodiment is a medication delivery apparatus 7, having a compartment 15 configured to accommodate a strip 1 of medication packages 9, the strip 1 for example configured on a roll such as a medication dose roll or configured in a stack of folded medication packages. Each package is provided with indicia (2b-d) indicating the time for consuming the medication of the package and/or the time for outputting the medication package from the medication delivery apparatus. Typically, these two times may be equivalent and the one approximated by the other as explained above in connection with the description of medication packages above. The medication delivery apparatus further comprises a medication package feeder mechanism (16) configured to transport medication packages from the compartment (15) to an outlet (5) of the medication delivery apparatus; a reader (4) configured to read said indicia of a medication package being transported by the medication package feeder mechanism; and a control unit (18) configured to control the medication delivery apparatus to output a medication package via the outlet at a time corresponding to the time for consuming as indicated by said indicia. This embodiment further comprises a sensor (20) configured to detect removal of an output medication packet (3a) from the outlet (5), and the medication delivery apparatus is configured to activate an alert signal, e.g. an alarm signal, when a preset time period from the indicated time for consuming has passed without the sensor (20) having detected that an output medication package has been removed from the outlet.

In variants of this embodiment, the reader (4) is a camera and the indicia (2b-d) indicating time for consuming the medication of the package is information printed on a label of the medication package. In further variants of this embodiments, the control unit (18) has access to information of a time schedule for outputting medication packages and wherein the medication delivery apparatus is configured to activate an alert signal or an alarm signal if the time for consuming the medication indicated on a medication package deviates from the corresponding time schedule information. The medication delivery apparatus may be configured to prevent outputting of a medication package if the information on the medication package deviates from the time schedule information, for example more than a preset deviation margin. In other variants, the medication delivery apparatus may be configured to control the outputting of a medication package based on either the indicia on the medication package or the time schedule information and activate an alert signal or an alarm signal for example to monitoring or nursing staff if the time information deviates.

Embodiments of the medication delivery apparatus may be configured with one, two or more compartments for separate medication package strips, each in a medication package roll or in a medication package stack. For example, a first medication package strip may have packages of a first set of pharmaceuticals and a second medication package strip may have packages of a second set of pharmaceuticals. For example, the packages of a first medication package strip may have indicia giving information for consumption or output of the package from the apparatus, and the packages of a second medication package strip may have indicia allowing the medication delivery apparatus to output a medication package based at least partly on for example sensor data, scheduled data or by remote control for example from a backend monitoring/control system.

In embodiments, the medication delivery apparatus is configured to activate an acoustic or visual attention signal when a medication package has been output and is available for the patient, in order to get the attention of the patient to take the dose of pharmaceuticals. The acoustic signal may for example be emitted from an alarm clock or a visual signal may be emitted from a light emitter, which may be integrated with the medication delivery apparatus or which may be external units coupled to and controlled by the control unit 18. A medication package sensor 20, for example in the form of an optical sensor, is provided at the output 5 and configured to detect the presence of an output medication package.

The sensor 20 is coupled to the control unit 18, which is configured to receive the sensor signal and monitor the presence and the removal of an output medication package from the outlet 5. When an output medication package has been removed from the outlet and thus been taken by a patient, the attention signal is deactivated by the control unit 18. When the control unit 18 has detected the removal of a medication package it may store information about this in an internal data memory and/or communicate this information to an external database.

Similarly, as in embodiments, when the control unit 18 detects that a medication package has not been removed within a preset time period, the control unit 18 may store information about this fact internally or communicate this for storage in an external database. The control unit 18 may also detect that a certain medication package should have been output at an earlier or later point in time, may then store or communicate this information in a similar manner.

The control unit 18 may be configured to send or activate an alert signal or an alarm locally close to the medical delivery apparatus and/or externally to an operator or nursing persons in such exemplified and other detected situations.

The medication strip compartment 15 is separated from the machinery compartment 14 by a partition 17 in the form of an interior wall with an access to the feeder mechanism allowing a medication package strip to be entered to the feeder mechanism. The medication strip compartment 15 is accessible from the exterior via a lid 19 of the housing 13. The lid 19 is provided with a lock mechanism 22, preferably coupled to the control unit 18. In preferred embodiments, the lock mechanism 22 of the lid may only be accessed by means of a separate electronic control device 30 such that the lid is prevented from being opened manually. This control device may be provided as a dedicated control device or a dedicated application program installed on a general computing device such as a smartphone or similar communicating with the control unit 18. The control device and/or application program is then configured to communicate control commands preferably wirelessly with the control unit 18 to control locking and/or unlocking of the lock mechanism by remote control from the exterior of the medication. In one embodiment the medication delivery apparatus may be provided with a connection port, for example a USB port or similar to connect a separate electronic control device or other peripheral devices to the control unit 18 of the medication delivery apparatus.

With this configuration, the lock mechanism is unlockable only from the interior of the medication delivery apparatus and cannot be operated manually from the outside of the housing. Overall, the medication delivery apparatus is configured such that it is operated and/or made accessible only from the interior of the housing (13), preferably by means of a pre-programmed or remote controlled control unit (18) comprised in the medication delivery apparatus. The remote control may be effected via a control device carried out by an authorized user or from a backend data processing system, e.g. in a monitoring central, via a data communication system.

Embodiments of the medication delivery apparatus are further configured to communicate or activate an alert or an alarm, at the site or via the data communications network to a remote receiver, for example at attempts of unauthorized opening of the lid, at breakage of the housing or the lid, at rough handling of the apparatus, sudden movements or unauthorized change of position as detected by a suitable sensor coupled to the control unit 18, such as accelerometers and/or vibration sensor and/or position sensor or the like. Similarly, a tracking or positioning device comprised in the medication delivery apparatus may be devised to render the apparatus possible to track when displaced. In a further developed embodiment, a position guard mechanism may be arranged to monitor that the medication delivery apparatus stays within a defined area for example by means of a geofence or by registering the proximity to or passing by a sensor device such as a NFC device configured to register entrance or departure from an apartment or rooms of a patient.

With this configuration of the housing 13 and the lid 19, the pharmaceuticals is protected in the interior and only the pharmaceuticals of a medication package output via the outlet 5 is available to the patient. The general idea of embodiments described herein is reduce and simplify the interfaces of the medication delivery apparatus such that a patient only needs pay attention to the outlet where a medication package is served at given points in time based on the indicia on the medication packages and/or a time schedule. A correct dose is dispensed from the apparatus without any required action from the patient or from caring staff. This greatly enhances patient safety with these types of medication delivery apparatus.

Further developments of the above embodiments may be configured for monitoring the status of a patient. Information regarding different types of personal data, here also called patient specific data, may indicate the daily fitness of a patient. Such patient specific data may for example be related to the movement pattern, the body weight, the body temperature or the blood glucose level of the patient. When a patient is on medication with pharmaceuticals here also generally called drugs, some expected behavior of patient specific data patterns related to the intake of drugs may be noticed. For example, drugs may be soporific, or exhilarant and cause a change of movement patterns, or a drug may cause the body temperature or the blood glucose level to change.

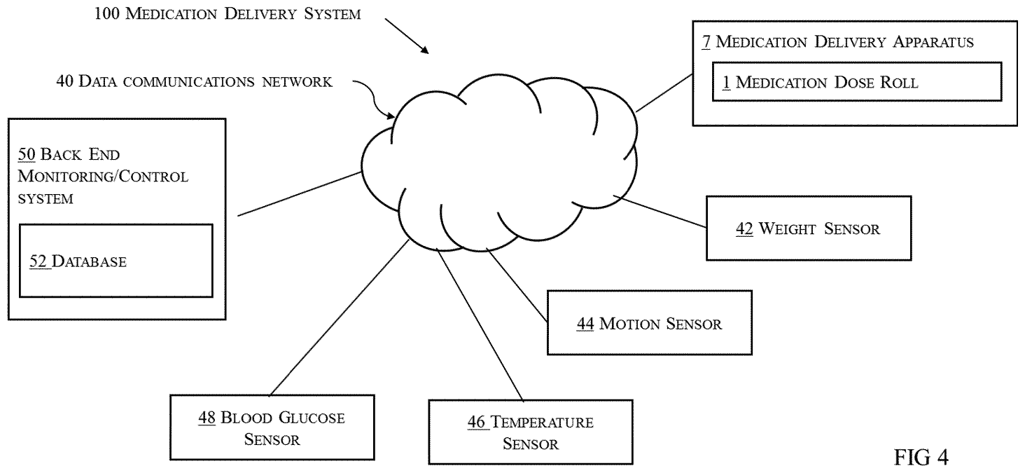

FIG. 4 shows embodiments of a medication delivery system 100 with a medication delivery apparatus 7 loaded with a strip of medication packages 1 on a medication dose roll and being communicatively coupled to other devices by means of a data communications network 40 as described above. A back end monitoring and/or control system 50 and a database 52 are communicatively coupled to the medication delivery apparatus 7 as well as different types of sensors may supply the medication delivery system with information regarding patient specific data of the patient. Such sensors may for example be a selection of a weight sensor 42, a motion sensor 44, a temperature sensor 46, proximity sensor, a temperature sensor (46), a continence sensor, a blood pressure sensor and/or a blood glucose sensor 48.

One or more of such sensors may be configured to gather patient specific data continuously, in periods, in connection with certain activities of the patient or at certain occasions possibly prompted by a signal from the medication delivery apparatus. Sensor data is communicated to the medication delivery apparatus and/or to the back end monitoring/control system. The sensor data may be processed to detect a selection of a sensor data pattern that for example deviates from a normal pattern of the patient, a predicted pattern of the patient due to medication, dose of medication, time of taking the medication or other factors. In case of deviation or detected events an alert or an alarm may be activated and communicated to a relevant receiver.

For example, if a patient gains weight, or loses weight, this may be an indication of an impaired health status. Monitoring the weight of a patient on a regular basis is therefore one method of early indicating changes of the health status of the patient. Monitoring weight may for example be performed by a weight sensor incorporated in the toilet seat at home for patients with home care. The weight sensor may as described be arranged to communicate with the medication delivery system, and if the weight of the patient deviates from a predicted span, a warning, an alert or an alarm signal is generated.

For some type of drugs or specific combination of drugs, a weight gain or loss may be expected. Still further, changes of the dosage of a drug may also result in an expected weight gain or loss. Information regarding patient specific type of drugs, or drug combinations, and the dosages thereof may be stored and made available in the system. So, for example the specification of each medication package that has been output from the medication delivery apparatus 7 to a patient may be stored in the back end database 52 and/or in a local memory of the medication delivery apparatus 7. The results from a weight monitoring process may for such cases be compared to a predicted and pre-stored development of an expected weight gain or loss and a warning signal will be generated if the result deviates from an expected development. Thereby, early indications of changes of the health status of the patient is provided.

Data from a motion sensor 44 and/or a temperature sensor 46 and/or other sensors may be used in a similar manner.

For patients with diabetes, the blood glucose levels are critical. The drug delivery system may be provided with a blood glucose meter or sensor 48 enabling the patient to take a blood test. The result of the blood test may be compared to patient specific information regarding the type of drugs, dosages of drugs and the time at which a drug, or combination of drugs, was last taken. The result can then be compared to a calculated, expected value. Deviations between the actual value and the calculated value may for example indicate that the dosage of the drug, or that the time span between subsequent intakes of drugs need to be adjusted. In one embodiment, the medication delivery system and/or the medication delivery apparatus may be configured to adjust the time between the output of two subsequent medication packages at least partly based on blood sensor data.

Different control features as described above may be executed locally in a medication delivery apparatus 7 or in a backend monitoring/control system 50, or in a combination thereof.

1. A medication delivery apparatus, comprising:
a housing configured to accommodate a strip of medication packages in its interior and having an outlet for outputting medication packages from the interior of the housing;
a lid of the housing configured to enable access to the interior of the housing;
a lock mechanism positioned in the interior of the housing and configured to lock the lid in a closed position;
wherein the medication delivery apparatus further comprises, or is communicatively coupled to, one or more sensors arranged to provide patient specific data.

2. The medication delivery apparatus of claim 1, wherein the one or more sensors is any of a weight sensor, a motion sensor, a temperature sensor, a proximity sensor, a temperature sensor, a continence sensor, a blood pressure sensor or a blood glucose sensor.

3. The medication delivery apparatus of claim 1, wherein data from the one or more sensors is processed to detect a selection of a sensor data pattern, and compared to one or more predicted sensor patterns of the patient, and wherein the medication delivery apparatus is configured to activate an alarm if the detected sensor pattern, or patterns, deviate from the predetermined pattern or patterns.

4. The medication delivery apparatus of claim 1, wherein the lock mechanism is electronically controllable, and is unlockable only from the interior of the medication delivery apparatus.

5. The medication delivery apparatus of claim 1, being configured such that it is operated and/or made accessible only from the interior of the housing, for example by means of a pre-programmed or remote controlled control unit comprised in the medication delivery apparatus.

6. The medication delivery apparatus of claim 1, having:
a compartment configured to accommodate a strip of medication packages 9, each package being provided with indicia (2b-d) indicating time for consuming the medication of the package;
a medication package feeder mechanism configured to transport medication packages from the compartment to an outlet of the medication delivery apparatus;
a reader configured to read said indicia of a medication package being transported by the medication package feeder mechanism;
a control unit configured to control the medication delivery apparatus to output a medication package via the outlet at a time corresponding to the time for consuming as indicated by said indicia, comprising a sensor configured to detect removal of an output medication packet from the outlet, wherein the medication delivery apparatus is configured to activate an alarm signal when a preset time period from the indicated time for consuming has passed without the sensor having detected that an output medication package has been removed from the outlet.

7. The medication delivery apparatus of claim 6, wherein the control unit have a digital data processor with a memory and one or more of a communication equipment for data communication via wired or wireless internet, such as with a wifi communication unit, telecommunication such as with a GSM unit with a SIM, short range radio communication, or Bluetooth, or infrared light with external units such as an external database, sensors or other periphery equipment.

8. The medication delivery apparatus of claim 6, wherein the digital data processor is equipped with an image processing system configured to interpret indicia in plain text OCR, Optical Character Recognition technology, bar code or QR code interpreting technology.

9. The medication delivery apparatus of claim 6, wherein the reader is an RFID reader configured to read and interpret indicia on an RFID tag or an NFC configured to read and interpret an NFC (Near Field Communication) chip.

10. The medication delivery apparatus of claim 6, wherein the reader is a camera and the indicia indicating time for consuming the medication of the package is information printed on a label of the medication package.

11. The medication delivery apparatus of claim 6, wherein the indicia on the medication packages is in the form of a printed bar code, or a QR code, or an RFID tag, or a NFC tag.

12. The medication delivery apparatus of claim 7, wherein the control unit has access to information of a time schedule for outputting medication packages and wherein the medication delivery apparatus is configured to activate an alarm signal if the time for consuming the medication indicated on a medication package deviates from the corresponding time schedule information.

13. The medication delivery apparatus of claim 1, further being configured to control the medication delivery apparatus to output one or more medication packages via the outlet at a predetermined time, possibly at least partly based on indicia of the one or more medication packages.

14. The medication delivery apparatus of claim 1, further being configured to compare patient specific data from one or more sensors with one or more pre-stored or calculated patient specific data values.

15. The medication delivery apparatus of claim 1, further being configured to generate an alert signal if said provided patient specific data deviates from said one or more predetermined patient specific data values.

16. The medication delivery apparatus of claim 1, further being configured to deliver medication packages with a predetermined time interval, and wherein said time interval is adjusted based at least partly based on sensor data and/or if an alert signal is generated.

17. The medication delivery apparatus of claim 1, further being configured to deliver a predetermined number of medication packages, and wherein the number of medication packages is adjusted at least partly based on sensor data and/or if an alert signal is generated.

18. A medication delivery system, comprising
a medication delivery apparatus configured to house a strip 1 of medication packages; and
a back end monitoring and/or control system;
wherein the medication package strip storage of the medication delivery apparatus may only be accessed by control from the exterior of the medication delivery apparatus by a control device.

19. A medication delivery system, further comprising the medication delivery apparatus of claim 1.
